(19) United States
(12) Patent Application Publication
(10) Pub. No.: US 2023/0203604 A1
(43) Pub. Date: Jun. 29, 2023

(54) DETECTION OF COVID-19 ASSOCIATED CARDIAC INJURY AND VACCINE-ASSOCIATED MYOCARDITIS

(71) Applicant: nference, Inc., Cambridge, MA (US)

(72) Inventors: Venkataramanan Soundararajan, Andover, MA (US); Aiveliagaram Venkatakrishnan, Woburn, MA (US)

(21) Appl. No.: 17/992,283

(22) Filed: Nov. 22, 2022

Related U.S. Application Data
(60) Provisional application No. 63/282,464, filed on Nov. 23, 2021.

(51) Int. Cl. C12Q 1/70; C12Q 1/6883

(52) U.S. Cl. CPC C12Q 1/701; C12Q 1/6883; C12Q 1/6869

(57) ABSTRACT

This application relates to methods of treating and/or preventing a coronavirus infection in a subject in need thereof, and treating cardiac injury in said subjects.

### CROSS REFERENCE TO RELATED APPLICATIONS

This application claims the benefit of U.S. Provisional Application No. 63/282,464, filed Nov. 23, 2021, which is hereby incorporated by reference in its entirety.

### BACKGROUND

Acute cardiac injury has been observed in a subset of COVID-19 patients, but the molecular basis for this clinical phenotype is unknown.

Cardiac injury is a prevalent complication associated with COVID-19.¹ In a study of 100 recently recovered COVID-19 patients, cardiovascular magnetic resonance imaging revealed cardiac involvement or ongoing myocardial inflammation in 78 and 60 patients, respectively.² In another study of 39 consecutive autopsies from patients who died of COVID-19, viral RNA was detectable in the heart of 24 (62%) patients. A large nationwide study from Israel on SARS-CoV-2 infected individuals reported increased association to cardiac disorders such as myocarditis, arrhythmia, myocardial infarction, and pericarditis.³ Myocarditis has also been reported in a small fraction of individuals after receiving an mRNA COVID-19 vaccine.⁴⁻⁶ The pathogenic mechanism of these complications remains unclear.

In the case of myocarditis associated to either COVID-19 infection or vaccination, a prevalent hypothesis posits that T lymphocytes and or antibodies that recognize SARS-CoV-2 antigens and mediate virus neutralization may also cross-react against cardiac proteins and trigger an autoimmune response against cardiomyocytes. This mechanism known as molecular mimicry has been suggested as a basis for myocarditis and other inflammatory conditions seen in the context of COVID-19 infection and vaccinations.⁷⁻¹⁰ Indeed, autoimmune sequelae of other infectious diseases have been attributed to mimicry between host and microbial antigens.¹¹⁻¹⁶

Such mimicry may contribute to cardiac inflammation during or after COVID-19 illness and warrants further experimental evaluation. SARS-CoV-2 variants harboring peptides identical to human cardiac proteins should be investigated as ‘viral variants of cardiac interest’.

### SUMMARY

In some aspects of the invention, provided herein are methods of treating a coronavirus infection in a subject in need thereof. Embodiments of the invention may comprise determining whether the subject is susceptible to an autoimmune inflammatory reaction by i) identifying candidate genes that are overexpressed in a target tissue or organ in the subject; ii) preparing a plurality of 8-mer peptide sequences encoded by the overexpressed candidate genes; iii) identifying at least one 8-mer peptide of the overexpressed candidate genes that is an identical match to a corresponding 8-mer viral peptide; and administering to said subject an autoimmune therapeutic. In some embodiments, said methods may comprise identifying the presence of at least one mimicked peptide set forth in Table 2, mutated cardiac peptide set forth in Table 3, or any combination thereof in a sample from the subject; and administering to said subject a therapeutic to the subject.

Other aspects of the invention provided herein include methods of treating or preventing cardiac injury in a subject infected with coronavirus. Such methods may comprise identifying the presence of at least one mimicked peptide set forth in Table 2, mutated cardiac peptide set forth in Table 3, or any combination thereof in a sample from the subject. In some embodiments, the presence of any one or more of the peptides set forth in Table 2 or mutated cardiac peptides set forth in Table 3 indicates whether the subject is susceptible to cardiac injury, and treating said patient for cardiac injury.

### BRIEF DESCRIPTION OF THE DRAWINGS

FIG. 1A and B depict the identification of mimicked peptides between SARS-CoV-2 and human proteins. A. Identification of cardiac specific proteins based on analysis of bulk RNAseq and single-cell RNAseq data and identification of SARS-CoV-2 proteins. B. Comparison of peptide libraries of human cardiac proteins and SARS-CoV-2 proteins.

FIG. 2 depicts the observed probability distribution for the Hamming distance between all 8-mers in the SARS-CoV-2 proteome and all 8-mers in reference sets of human proteins. Shown is the distribution of Hamming distance between all SARS-CoV-2 linear 8-mer peptides (n=9,926 peptides) and all linear 8-mer peptides from canonical isoforms of human cardiac proteins (orange, n=129,415 peptides), brain proteins (blue, n=241,046 peptides), and skin proteins (green, n=88,628 peptides). Error bars represent 95% confidence intervals.

### DETAILED DESCRIPTION

It has been hypothesized that molecular mimicry may play a role in triggering an autoimmune inflammatory reaction in some individuals after SARS-CoV-2 infection. Disclosed herein is the analysis of linear peptides contained in proteins that are primarily expressed in the heart. Said proteins may occur in the SARS-CoV-2 proteome. Advances in next-generation sequencing technologies facilitated the rapid development of large-scale multi-omic datasets and genomic epidemiology resources to better understand the COVID-19 pandemic. Bulk and single cell RNA-sequencing datasets elucidated the transcriptional signatures of most healthy human tissues and cell types.¹⁷⁻²⁰ Amino acid sequences of human proteins, including genetic variants and immunologic epitopes, are available in UniProt²¹, gnomAD²² and Immune Epitope Database (IEDB)²³. Finally, the GISAID database currently hosts 4.85 million SARS-CoV-2 genomes from more than 200 countries.²⁴ The availability of such genome-scale data was exploited to investigate the potential for molecular mimicry between SARS-CoV-2 and human cardiac proteins.

A systematic comparison of peptides from human cardiac proteins and SARS-CoV-2 proteins was conducted. No 8-mer peptides were identical between the reference sequences of these two groups of proteins. However, when including human and viral genetic variants in this comparison, 21 8-mer peptides were found to be identical between human cardiac proteins and SARS-CoV-2 proteins. Among these, a human genetic variant of Myosin Heavy Chain 6 (MYH6) (c.5410C>A; Q1804K) is identical to a peptide of the reference SARS-CoV-2 replicase polyprotein. The SARS-CoV-2 variants that have peptides identical to human cardiac proteins should be assessed as potential ‘variants of cardiovascular interest’.

Specifically, as disclosed herein, a library of 136,704 8-mer peptides from 144 human proteins (including splicing variants) were compared to 9,926 8-mers from all 17 viral proteins in the reference SARS-CoV-2 proteome. No 8-mers were identical between the reference human proteome and the reference SARS-CoV-2 proteome. However, there were 45 8-mers that differed by only one amino acid when compared to the reference SARS-CoV-2 proteome. Analysis of protein-coding mutations from 141,456 individuals showed that one of these 8-mers from the SARS-CoV-2 Replicase polyprotein 1a/1ab (KIALKGGK) is identical to a mutant MYH6 peptide encoded by the c.5410C>A (Q1804K) genetic variant, which has been observed at low prevalence in Africans/African Americans (0.08%), East Asians (0.3%), South Asians (0.06%) and Latino/Admixed Americans (0.003%). Furthermore, analysis of 4.85 million SARS-CoV-2 genomes from over 200 countries shows that viral evolution has already resulted in 20 additional 8-mer peptides that are identical to human heart-enriched proteins encoded by reference sequences or genetic variants.

In some aspects of the invention, provided herein are methods of treating a coronavirus infection in a subject in need thereof. Embodiments of the invention may comprise determining whether the subject is susceptible to an autoimmune inflammatory reaction by i) identifying candidate genes that are overexpressed in a target tissue or organ in the subject; ii) preparing a plurality of 8-mer peptide sequences encoded by the overexpressed candidate genes; iii) identifying at least one 8-mer peptide of the overexpressed candidate genes that is an identical match to a corresponding 8-mer viral peptide; and administering to said subject an autoimmune therapeutic.

Other aspects of the invention provided herein include methods of treating or preventing cardiac injury in a subject infected with coronavirus. Such methods may comprise identifying the presence of at least one mimicked peptide set forth in Table 2, mutated cardiac peptide set forth in Table 3, or any combination thereof in a sample from the subject. In some embodiments, the presence of any one or more of the peptides set forth in Table 2 or mutated cardiac peptides set forth in Table 3 indicates whether the subject is susceptible to cardiac injury, and treating said patient for cardiac injury.

In some aspects of the invention, provided herein are methods of treating a coronavirus infection in a subject in need thereof, comprising identifying the presence of at least one mimicked peptide set forth in Table 2, mutated cardiac peptide set forth in Table 3, or any combination thereof in a sample from the subject; and administering to said subject a therapeutic to the subject.

In some embodiments of the disclosed invention, the therapeutic administered to the subject is selected from remdesivir, antibody therapy, dexamethasone, convalescent plasma, ritonavir, and molnupiravir. In some such embodiments, the antibody therapy comprises one or more of sotrovimab, bamlanivimab, etesevimab, casirivimab, and imdevimab.

### EXAMPLES

### Example 1: Identification of Genes that are Overexpressed in Cardiac Tissue

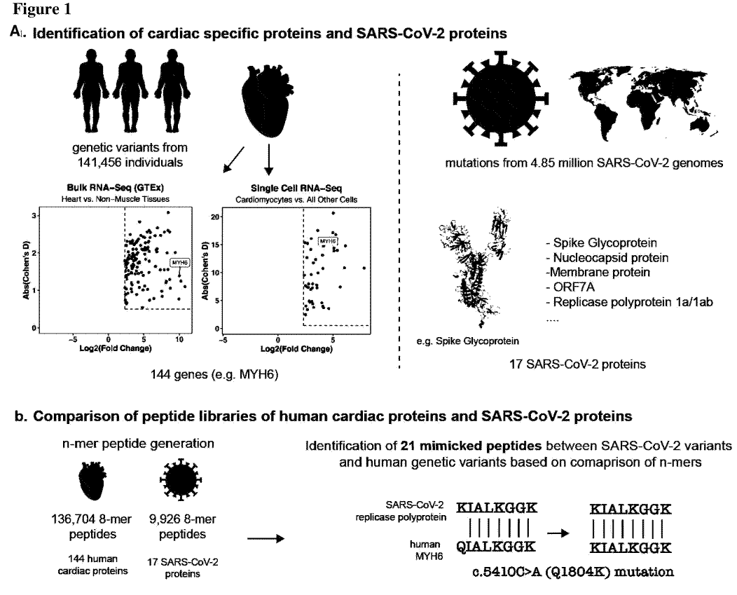

To identify heart-enriched proteins, the expression of all human protein coding genes in heart samples (n = 861) were compared to all non-striated muscle samples (n = 15,718) from the Genotype Tissue Expression (GTEx) project. There were 137 genes expressed at least 5-fold higher in heart with a Cohen’s D value greater than or equal to 0.5 (FIG. 1, Table 4). Similarly, the expression of all genes between cardiomyocytes (n = 8.9 K cells) and non-cardiomyocytes (n = ~2.5 million cells) were compared across a database of 52 single cell RNA-sequencing studies covering 62 tissues.¹⁸ There were 46 genes overexpressed in cardiomyocytes based on the same criteria outlined above (FIG. 1, Table 4). Combining the lists of genes identified from bulk and single cell RNA-sequencing analyses, resulted in a total of 144 candidate cardiac proteins.

### Example 2: MYH6 Variant is Mimicked by an Epitope of SARS-CoV-2 Replicase Polyprotein 1a/1ab

Peptide 8-mers were computed from the reference sequences of the 144 cardiac proteins (including isoforms) and all 17 proteins from the reference SARS-CoV-2 sequence. The pairwise sequence identity was then systematically compared (using Hamming Distance; see Methods) for the 136,704 cardiac protein 8-mers with the 9,926 8-mers derived from the SARS-CoV-2 proteins. No peptides were identical between these two groups. However, 45 8-mers were nearly identical, with only a single mismatched amino acid (Table 1). To determine whether human genetic variation results in any 8-mers which exactly match the reference SARS-CoV-2 proteome, amino acid mutations from 141,456 individuals were then analyzed using the gnomAD database (including 83,623 mutations in the 144 cardiac proteins).²²

One specific 8-mer from the SARS-CoV-2 Replicase polyprotein 1a/1ab (KIALKGGK) was identical to a mutant peptide encoded by the c.5410C>A(Gln1804Lys) variant in human MYH6 that results in (KIALKGGK). This variant can be identified in Africans/African Americans (0.08% prevalence), East Asians (0.3% prevalence), South Asians (0.06% prevalence) and Latino/Admixed Americans (0.003% prevalence). Analysis of peptides from IEDB shows that the non-mutated 7-mer in this peptide (IALKGGK) also overlaps with a known B-cell epitope from SARS-CoV-2 (IALKGGKIVNNWLKQ)²⁵. Whether the mimicry between SARS-CoV-2 Replicase polyprotein 1a/1ab and wild-type or mutant MYH6 contributes to cardiac inflammation in the setting of COVID-19 warrants further investigation.

TABLE 1

List of peptide pairs from SARS-CoV-2 proteins and human cardiac proteins that have a Hamming distance less than or equal to 1

| SARS-CoV-2 Proteins | | | Human Cardiac-Enriched Proteins | | |
|---|---|---|---|---|---|
| Protein Name | Amino Acid Positions | Amino Acid Sequence | Protein Name | Isoforms Containing Sequence | Amino Acid Sequence |
| Spike glycoprotein WT/2P | 491-498 | PLQSYGFQ | KLHL41 | 1,2 | PLQSYFFQ |
| Spike glycoprotein WT/2P | 856-863 | NGLTVLPP | FHOD3 | 1, 2, 3, 4 | IGLTVLPP |
| Spike glycoprotein WT/2P | 857-864 | GLTVLPPL | FHOD3 | 1, 2, 3, 4 | GLTVLPPP |
| Spike glycoprotein WT/2P | 1087-1094 | AHFPREGV | CMYA5 | 1 | AHFPAEGV |
| Replicase polyprotein lab | 4937-4944 | KYAISAKN | TTN | 1, 3, 2, 5, 12, 13, 7, 8, 4, 9, 10, 11 | KYIISAKN |
| Replicase polyprotein lab | 5604-5611 | LQGPPGTG | MYLK3 | 1, 4 | KQGPPGTG |
| Replicase polyprotein 1ab | 5605-5612 | QGPPGTGK | MYLK3 | 1, 4 | QGPPGTGR |
| Replicase polyprotein 1ab | 5813-5820 | NRPQIGVV | CASQ2 | 1, 2 | FRPQIGVV |
| Replicase polyprotein 1ab | 5814-5821 | RPQIGVVR | CASQ2 | 1, 2 | RPQIGVVN |
| Replicase polyprotein 1ab | 5955-5962 | DTKFKTEG | TTN | 1, 3, 2, 5, 12, 13, 7, 8, 4, 9, 10, 11 | DTKFKTTG |
| Replicase polyprotein 1ab | 5955-5963 | DTKFKTEGL | TTN | 1, 3, 2, 5, 12, 13, 7, 8, 4, 9, 10, 11 | DTKFKTTGL |
| Replicase polyprotein 1ab | 5956-5963 | TKFKTEGL | TTN | 1, 3, 2, 5, 12, 13, 7, 8, 4, 9, 10, 11 | TKFKTTGL |
| Replicase polyprotein 1ab | 6516-6523 | KPVPEVKI | CMYA5 | 1 | KPSPEVKI |
| Replicase polyprotein 1ab | 6516-6523 | KPVPEVKI | TTN | 1, 2, 5, 12, 13, 7, 8, 4, 11 | KPVPEEKI |
| Replicase polyprotein la/lab | 207-214 | RAGKASCT | TTN | 1, 2, 5, 12, 13, 7, 8, 4, 11 | EAGKASCT |
| Replicase polyprotein la/lab | 208-215 | AGKASCTL | TTN | 1, 2, 5, 12, 13, 7, 8, 4, 11 | AGKASCTT |
| Replicase polyprotein la/lab | 345-352 | GTENLTKE | TNNI3K | 1, 3, 4 | GTESLTKE |
| Replicase polyprotein la/lab | 459-466 | VNINIVGD | ENO3 | 1, 3, 2 | VNIQIVGD |
| Replicase polyprotein la/lab | 492-499 | KGLDYKAF | CASQ2 | 2 | KKLDYKAF |
| Replicase polyprotein la/lab | 512-519 | TKGKAKKG | MYH7 | 1 | GKGKAKKG |
| Replicase polyprotein la/lab | 513-520 | KGKAKKGA | MYH7 | 1 | KGKAKKGS |
| Replicase polyprotein la/lab | 879-886 | VIKTLQPV | LMOD3 | 1 | VIKTLKPV |
| Replicase polyprotein la/lab | 963-970 | GATSAALQ | ANKRD2 | 1, 2 | GAQSAALQ |
| Replicase polyprotein la/lab | 1143-1150 | VLLAPLLS | HJV | 1,2,3 | TLLAPLLS |
| Replicase polyprotein la/lab | 1144-1151 | LLAPLLSA | HJV | 1,2,3 | LLAPLLSG |
| Replicase polyprotein la/lab | 1197-1204 | KQVEQKIA | GOT1 | 1, 2 | KKVEQKIA |
| Replicase polyprotein la/lab | 2246-2253 | STAALGVL | SLC4A3 | 1,2,3 | STAVLGVL |
| Replicase polyprotein la/lab | 2533-2540 | KGSLPINV | TTN | 1, 2, 5, 12, 13, 7, 9, 4, 11 | KGSLPITV |
| Replicase polyprotein la/lab | 2550-2557 | EESSAKSA | MYH7B | 1 | EESKAKSA |
| Replicase polyprotein la/lab | 2630-2637 | LSTFISAA | TENM2 | 1, 2 | LSTFFSAA |
| Replicase polyprotein la/lab | 2757-2764 | KIALKGGK | MYH6 | 1 | QIALKGGK |
| Replicase polyprotein la/lab | 2757-2764 | KIALKGGK | MYH7 | 1 | QIALKGGK |
| Replicase polyprotein la/lab | 2758-2765 | IALKGGKI | MYH6 | 1 | IALKGGKK |
| Replicase polyprotein la/lab | 2758-2765 | IALKGGKI | MYH7 | 1 | IALKGGKK |
| Replicase polyprotein la/lab | 3908-3915 | FEKMVSLL | MYH6 | 1 | EEKMVSLL |
| Replicase polyprotein la/lab | 3908-3915 | FEKMVSLL | MYH7 | 1 | EEKMVSLL |
| Replicase polyprotein la/lab | 3909-3916 | EKMVSLLS | MYH6 | 1 | EKMVSLLQ |
| Replicase polyprotein la/lab | 3909-3916 | EKMVSLLS | MYH7 | 1 | EKMVSLLQ |
| Replicase polyprotein la/lab | 4137-4144 | VKLQNNEL | TBX20 | 1 | VKLTNNEL |
| Putative ORF9c protein | 47-54 | AAVGELLL | ASB10 | 1,2,3 | AAVVELLL |
| ORF7b protein | 14-21 | LAFLLFLV | TMEM182 | 2, 1, 3 | LAGLLFLV |
| ORF7a protein | 43-50 | NSPFHPLA | FLNC | 1, 2 | NSPFHVLA |
| Nucleoprotein | 192-199 | NSSRNSTP | CMYA5 | 1 | NSSRSSTP |
| Nucleoprotein | 374-381 | KKADETQA | MYPN | 1 | EKADETQA |
| Nucleoprotein | 375-382 | KADETQAL | MYPN | 1 | KADETQAR |

### Example 3: SARS-CoV-2 Variants Harbor Epitopes that are Identical to Peptides of Cardiac Proteins

Whether SARS-CoV-2 evolution has given rise to variants that harbor peptides identical to human cardiac proteins was next examined. Analysis of 4.85 million SARS-CoV-2 genomes from over 200 countries obtained from the GISAID database was conducted.²⁴ Twenty-one 8-mer peptides from SARS-CoV-2 variants were identified as identical to cardiac proteins encoded by reference sequences or genetic variants (Tables 2 and 3). Of these 21 peptides, the one present in the highest number of viral sequences was STAVLGVL, which mapped to the NSP3 protein in 4,501 (0.09%) SARS-CoV-2 genomes. This exact sequence is present in a transmembrane helix of all three described isoforms of the cardiac protein SLC4A3, an anion exchange protein that is associated with short QT syndrome (Tables 2 and 3).^{26,27} Although no epitopes containing this full sequence were present in IEDB, relaxing the match to 90% similarity using BLAST²⁸ identified a similar epitope (HEAQAVLGVLL) that is reported to bind to both HLA-B*40:1 and HLA-B*58:01.

TABLE 2

List of identical cardiac peptides found in SARS-CoV-2 variants in GISAID. The NSP proteins are cleaved products of the replicase polyprotein

| No. of GISAID Entries with Identical Match | SARS-CoV-2 Gene | Mimicked Peptide | Approx. Star-End in SCOV2 Protein | Cardiac Protein | Cardiac Protein Start-End | Exact IEDB epitope match | Identical IEDB Epitope Seq (HLA/antigen infor) [>= 90% identity] |
|---|---|---|---|---|---|---|---|
| 4501 | NSP3 | STAVLGVL | 1429-1436 | SLC4A3 | 746-753 | No | HEAQAVLGVLL (HLA-B*40:01); |
| | | | | | | | HEAQAVLGVLL (HLA-B*40:01) |
| 1322 | NSP2 | GTESLTKE | 166-173 | TNNI3K | 294-301 | No | NA |
| 580 | N | NSSRSSTP | 193-200 | CMYA5 | 88-95 | No | TINSSRSSQESY (B-cell epitopes, MHC ligands) |
| 205 | NS9c | AAVVELLL | 48-55 | ASB10 | 307-314 | No | MVDPQLDGPQLAALAAVVELGSFDA () |
| 118 | NSP2 | KGKAKKGS | 334-341 | MYH7 | 635-642 | Yes | AGADAPIEKGKGKAKKGSS (MHC ligand) |
| 33 | NSP3 | VIKTLKPV | 60-67 | LMOD3 | 514-521 | No | KVAIKTLKPGTMS (HLA-A*02:01); |
| | | | | | | | TKVAIKTLKPGTMSPE (HLA-A*02:01) |
| 17 | NSP3 | KKVEQKIA | 380-387 | GOT1 | 55-62 | | GEKVEQKIEGKWVNEKKAQEDKLQ (MHC Class I, II, Bcell epitope) |
| 14 | NS7a | NSPFHVLA | 44-51 | FLNC | 1727-1734 | No | NA |
| 11 | N | EKADETQA | 375-382 | MYPN | 89-96 | No | ADETQALPQRQKKQQ (HLA Class II) |
| 9 | NSP3 | TLLAPLLS | 304-311 | HJV | 409-416 | | NFNQHEVLLAPLLS (Bcell epitope and MHC ligand) |
| 6 | NSP3 | LSTFFSAA | 1811-1818 | TENM2 | 1036-1043 | No | AGTLSTFFGVPLVLT (HLA class II MHC restriction) |
| 6 | NSP3 | LLAPLLSG | 327-334 | HJV | 410-417 | No | ENFNQHEVLLAPLLS (Bcell and MHC) |
| 6 | Spike | IGLTVLPP | 854-861 | FHOD3 | 971-978 | No | GFIKQYGDCLGDIAARDLICAQKFNGLTVLPPLLTDEMIAQYT (Tcell, Bcell and MHC ligands) |
| 2 | NSP13 | QGPPGTGR | 282-289 | MYLK3 | 373-380 | No | ILYGPPGTGK (HLA-A*03:01) |
| 1 | NSP15 | KPVPEEKI | 65-72 | TTN | 10277-10284 | No | EAPLYVVDKPVPEESE (HLA-RB1*04:01) |
| 1 | NSP3 | KGSLPITV | 1716-1722 | TTN | 5437-5444 | No | SLPITVYYAV (Tcell, Bcell and MHC ligands) |

TABLE 3

List of mutated cardiac peptide nmers from human genetic variants identical to SARS-CoV-2 variants. The NSP proteins are cleaved products of the replicase polyprotein

| Human Cardiac Gene | rsID | Mutation consequence | Mutated cardiac Peptide | Wild-type cardiac peptide | No. ofGISAID genomes | SARS- CoV-2 gene | IEDB epitope Exact match | IEDB epitope info (>=90 % Seq Identity) |
|---|---|---|---|---|---|---|---|---|
| FLNC | rs374848954 | p.Val1732Leu | NSPFHLLA | NSPFHVLA | 1661 | NS7a | Yes | NSPFH (HLA-A*01:01) |
| LMOD3 | rs370869958 | p.Lys519Arg | VIKTLRPV | VIKTLKPV | 85 | NSP3 | No | NA |
| TMEM182 | rs774398171 | p.Gly215Val | LAVLLFLV | LAGLLFLV | 21 | NS7b | No | NA |
| MYLK3 | rs771870674 | p.Arg380Cys | QGPPGTGC | QGPPGTG R | 1 | NSP13 | No | GPPGTGKSHFAIGLA (B cell, Tcell, MHC ligand) |

### Example 4: Discussion

The mechanistic basis of acute cardiac injury in COVID-19 patients remains unknown. Viral infections have previously been proposed to trigger autoimmune reactions, and it has been hypothesized that molecular mimicry plays a role in mediating autoimmune reactions.^{7-9,11-13,29} Here, a systematic analysis of gene-expression from cardiac tissues, based on bulk RNAseq data and single cell RNAseq data, combined with analysis of human genetic variation and SARS-CoV-2 genomes has led to the identification of candidate proteins and peptide regions that might be involved in immune cross reactivity (Tables 2 and 3). These newly identified identical peptides expand the set of known shared peptides between the human proteome and SARS-CoV-2, such as the furin cleavage site ‘RRARSVAS’ present in both human ENaC-α and the Spike glycoprotein.^{10,30,31} Further research is warranted to ascertain whether these mimicked peptides contribute to inflammation mediated by an autoimmune mechanism in context of COVID-19 infection. Moreover, in context of this report, the incidence of myocarditis observed in some vaccinated individuals demands scrutiny of potential molecular mimicry between the antigenic proteome selected for COVID-19 vaccines (e.g., pre-fusion stabilized Spike protein) and human recipients.^{4-6,32,33}

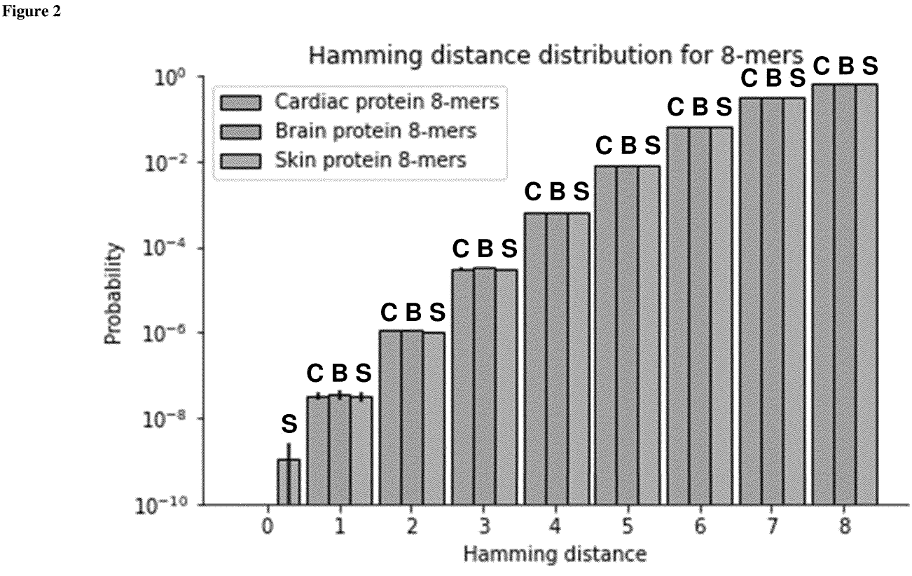

The human proteins surveyed were shortlisted based on their overexpression in cardiac tissue. There could be mimicked proteins that are shared between cardiac tissues and other tissues that are not accounted for in the analysis. There are other mechanisms that could explain autoimmune conditions after viral infection such as bystander activation, epitope spreading, and viral persistence. In bystander activation, nonspecific immune response to viral infection leads to release of self-antigens from affected tissue. Presentation of these self-antigens on antigen presenting cells can activate pre-primed autoreactive T cells. In epitope spreading, the inflammatory antiviral response leads to the killing of uninfected neighboring cells and release of more self-antigens. In persistent viral infections, long term presence of the viral particles may lead to a continued antiviral response and immunopathology.^{34,35} The presence of identical peptides in cardiac proteins and the SARS-CoV-2 proteome could occur due to chance. Comparing all SARS-CoV-2 8-mers to a set of brain enriched proteins and a set of skin enriched proteins (FIG. 2) shows a similar probability distribution of Hamming distance, suggesting that the observed similarity with SARS-CoV-2 peptides is not specific to human cardiac proteins. It is also possible that peptides with lower degrees of similarity could contribute to immunologic mimicry, as T cells can be highly cross reactive against different major histocompatibility complex (MHC)-presented peptides.³⁶⁻⁴⁰

Taken together, by studying the intersection of human genetic variation in cardiac proteins and SARS-CoV-2 evolution, candidates of molecular mimicry were identified that have potential to contribute to cardiac inflammation in the context of COVID-19. It will be important to perform follow-up functional studies evaluating the potential of SARS-CoV-2 reactive T cells and antibodies (e.g., from active or recovering COVID-19 patients) to cross-react with these peptides. Thus, it is proposed that SARS-CoV-2 variants harboring peptides identical to host heart-enriched proteins should be studied as ‘viral variants of cardiac interest’. A similar strategy can be applied to identify and categorize plausible mimicry candidates from any human tissues that are targeted by other autoimmune responses in COVID-19 patients.

### Example 5: Methods

### Identification of Proteins Enriched in Cardiac Tissue

Bulk RNA-sequencing (RNA-seq) data was accessed from the Genotype Tissue Expression (GTEx) project V8.¹⁷ For each sample, FASTQ files were processed using Salmon (in mapping-based mode) to quantify gene expression in transcripts per million (TPM). Specifically, the expression of each transcript isoform was first determined by passing FASTQ files to Salmon quant with the following parameters passed: validateMappings, rangeFactorizationBins 4, gcBias, biasSpeedSamp 10. All isoforms are then summed via a transcript-to-gene map, generating a gene-level expression value. GRCh38 was used as the reference, including cDNA and non-coding RNA.

For single cell RNA-seq studies, processed count matrices were accessed from Gene Expression Omnibus or other publicly available data repositories. There were two datasets analyzing heart tissues which captured cardiomyocytes, the main cell type of interest for this report.^{20,41} Other datasets captured a wide variety of immune, stromal, and parenchymal cell types from tissues including the respiratory tract, gastrointestinal tract, genitourinary tract, hepatobiliary system, skeletal muscle, brain, skin, eyes, and endocrine organs. Each dataset was processed using Scrublet and Seurat v3.0 as described previously.^{18,42-44} Cell type annotations were obtained from associated metadata files if available; otherwise, annotation was performed manually, guided by the cell types reported in the associated publication.

To identify genes that were overexpressed in cardiac tissue, fold change and Cohen’s D values was calculated between defined sample cohorts. For bulk RNA-seq data, Cohort A was defined as all GTEx heart samples (n = 861), and Cohort B was defined as all remaining GTEx samples except for those derived from skeletal muscle (n = 15,718). For single cell RNA-seq data, Cohort A was defined as all cells annotated as cardiomyocytes (n ~ 8900 cells), and Cohort B was defined as all other cells from all processed studies (n ~ 2.5 million cells). Fold change and Cohen’s D were calculated as follows:

$$\begin{matrix}F o l d C h a n g e = \frac{T P {M}_{C o h o r t A} + 1}{T P {M}_{C o h o r t B} + 1} \\ C o h e n ′ s D = \frac{T P {M}_{C o h o r t A} - T P {M}_{C o h o r t B}}{S {D}_{p o o l e d}}\end{matrix}$$

where the pooled standard deviation SD_{pooled} is defined as:

$$\begin{matrix}S {D}_{p o o l e d} = \\ \sqrt{\frac{\left({N}_{C o h o r t A} - 1\right) \times S {D}_{C o h o r t A}^{2} + \left({N}_{C o h o r t B} - 1\right) \times S {D}_{C o h o r t B}^{2}}{\left({N}_{C o h o r t A} + {N}_{C o h o r t B} - 2\right)}} ,\end{matrix}$$

where N_{Cohort A} and N_{Cohort B} are the number of samples in Cohorts A and B, respectively, and SD_{Cohort A} and SD_{Cohort B} are the standard deviation of TPM values for the given gene in Cohorts A and B, respectively.

Genes with Fold Change ≥ 5 and Cohen’s D ≥ 0.5 from either the bulk or single cell RNA-seq analysis were considered to be enriched in cardiac tissue. In the volcano plots used to visualize these analyses, genes were filtered to a TPM or CP10K value ≥ 1 in either Cohort A or Cohort B, and genes meeting the criteria for overexpression in heart or cardiomyocytes are colored in red.

For a control analysis, genes overexpressed in the brain or skin were also identified by bulk RNA-sequencing from the GTEx project. The same approach was used as described above, except that Cohort A was defined as either all brain samples (n = 2,351) or all skin samples (n = 1,305), and Cohort B was defined as all other samples.

TABLE 4

List of human cardiac proteins identified based on Bulk RNA-seq and Single cell RNA-seq

| Gene | Bulk RNAseq Cohen’sD | Bulk RNAseq Fold Change | Single cell RNAseq Cohen’sD | Single cell RNAseq Fold Change |
|---|---|---|---|---|
| NPPA | 1.1 | 1796.96 | 10.81 | 228.63 |
| MYL7 | 1.25 | 1206.31 | 13.98 | 41.82 |
| MYH6 | 1.39 | 1035.51 | 14.39 | 33.95 |
| NPPB | 0.77 | 978.15 | 7.39 | 3.08 |
| TNNI3 | 2.01 | 688.88 | 13.9 | 34.44 |
| MYL2 | 1.06 | 426.75 | 14.8 | 70.24 |
| MYH7 | 1.29 | 403.97 | 20.66 | 32.61 |
| MYBPC3 | 2.56 | 398.67 | 14.13 | 7.9 |
| TNNT2 | 3.08 | 349.01 | 15.97 | 40.17 |
| CSRP3 | 1.56 | 342.89 | 17.53 | 12.22 |
| MB | 2.6 | 339.57 | 10.75 | 16.91 |
| NMRK2 | 2 | 312.46 | 11.29 | 3.98 |
| MYL3 | 1.24 | 287.28 | 12.33 | 10.69 |
| MYL4 | 1.21 | 278.12 | 4.43 | 8.92 |
| COX6A2 | 2.56 | 238.14 | 13.13 | 16.49 |
| ANKRD1 | 1.55 | 236.39 | 16.81 | 38.11 |
| BMP10 | 0.84 | 192.77 | 0.02 | 1 |
| XIRP1 | 0.98 | 186.09 | 11.67 | 3.3 |
| TCAP | 2.47 | 165.66 | 11.75 | 9.32 |
| LMOD2 | 1.55 | 161.06 | 15.38 | 5.41 |
| CKM | 2.67 | 121.39 | 14.76 | 17.56 |
| TNNC1 | 2.11 | 116.52 | 9.95 | 12.41 |
| ACTC1 | 2.03 | 114.83 | 17.17 | 31.65 |
| TECRL | 2.08 | 108.63 | 15.24 | 6.41 |
| NRAP | 1.64 | 100.1 | 9.86 | 2.67 |
| MYOZ2 | 2.46 | 96.37 | 16.1 | 8.11 |
| ACTA1 | 0.78 | 85.4 | 7.79 | 11.48 |
| NKX2-5 | 2.55 | 60.66 | NA | NA |
| SMPX | 2.41 | 58.97 | 11.02 | 3.39 |
| ANKRD2 | 0.74 | 56.65 | 3.14 | 1.48 |
| MYOM2 | 2.04 | 54.35 | 3.28 | 2.9 |
| SYNPO2L | 1.78 | 54.28 | 7.88 | 3.21 |
| FABP3 | 2.6 | 53.66 | 4.9 | 7.11 |
| DHRS7C | 1.48 | 53.13 | 3.91 | 1.26 |
| HRC | 2.31 | 52.1 | 9.54 | 5.25 |
| ACTN2 | 2.29 | 49.24 | 7.42 | 6.34 |
| CKMT2 | 2.15 | 48.13 | 5.57 | 3.19 |
| TRIM63 | 2.11 | 46.3 | 3.35 | 1.75 |
| SLN | 1.03 | 45.97 | 4.25 | 3.84 |
| HSPB3 | 2.56 | 44.41 | 6.57 | 2.16 |
| FBXO40 | 2.08 | 38.61 | 8.3 | 2.2 |
| APOBEC2 | 1.83 | 38.37 | 2.02 | 1.33 |
| SRL | 2.27 | 37.54 | 5.02 | 1.85 |
| MYOM3 | 2.14 | 36.29 | 3.86 | 1.64 |
| UNC45B | 1.97 | 35.35 | 4.96 | 1.59 |
| TTN | 2.41 | 34.78 | 10.85 | 65.81 |
| SBK2 | 1.12 | 34.73 | 0.08 | 1 |
| MYLK3 | 2.08 | 30.66 | 3.38 | 1.59 |
| RPL3L | 1.4 | 30.36 | 3.51 | 1.44 |
| TRIM54 | 2.43 | 30.03 | 1.55 | 1.25 |
| MYBPHL | 1.01 | 29.58 | 3.8 | 1.64 |
| XIRP2 | 0.54 | 28.03 | 12.68 | 6.12 |
| CMYA5 | 2.1 | 26.54 | 7.28 | 11.68 |
| SMYD1 | 1.48 | 25.33 | 6.66 | 1.58 |
| FITM1 | 2.47 | 23.91 | 4.26 | 1.96 |
| SCN5A | 2.2 | 23.56 | 2.62 | 1.26 |
| TRDN | 2.83 | 21.15 | 7 | 4.6 |
| LMOD3 | 1.74 | 20.88 | 2.32 | 1.78 |
| MYPN | 1.76 | 20.84 | 3.81 | 1.39 |
| TRIM55 | 1.08 | 20.55 | 2.44 | 1.4 |
| LDB3 | 2.37 | 19.06 | 7.97 | 6.87 |
| SMCO1 | 1.45 | 18.71 | 4.26 | 1.25 |
| KLHL31 | 1.73 | 18.44 | 5.15 | 1.73 |
| LRRC14B | 1.22 | 18.39 | 0.07 | 1 |
| PXDNL | 2.01 | 17.72 | 0.72 | 1.13 |
| POPDC2 | 2.32 | 16.72 | 3.57 | 1.89 |
| PPP1R3A | 1.62 | 16.72 | 5.73 | 1.46 |
| ABRA | 0.89 | 15.75 | 5.75 | 1.63 |
| MYO18B | 1.96 | 15.7 | 3.25 | 1.38 |
| HHATL | 2.3 | 15.55 | 0.82 | 1.2 |
| LRRC10 | 1.52 | 15.46 | 3.59 | 1.13 |
| MYH7B | 2.1 | 15.44 | 1.42 | 1.29 |
| MYOM1 | 1.84 | 15.35 | 7.65 | 5.61 |
| SGCG | 2.28 | 15.18 | 2.37 | 1.45 |
| ENO3 | 1.84 | 14.26 | 4.15 | 2.92 |
| RYR2 | 1.81 | 14.09 | 4.02 | 5.77 |
| TBX20 | 1.44 | 13.73 | 0.06 | 1 |
| HSPB7 | 1.88 | 13.18 | 9.66 | 8.07 |
| ADPRHL1 | 2.24 | 13.08 | 1.81 | 1.49 |
| ALPK2 | 2.22 | 12.49 | 2.64 | 1.44 |
| ASB11 | 1.67 | 12.1 | 0.22 | 1.02 |
| CASQ2 | 1.93 | 11.98 | 7.04 | 3.65 |
| TMEM182 | 1.95 | 11.83 | 1.64 | 1.45 |
| PERM1 | 1.79 | 11.75 | 0.77 | 1.06 |
| ASB15 | 1.63 | 11.34 | 1.24 | 1.18 |
| TXLNB | 1.7 | 11.25 | 2.56 | 1.74 |
| MYZAP | 1.97 | 11.15 | 3.56 | 2.28 |
| ITGB1BP2 | 2.1 | 11.09 | 2.37 | 1.25 |
| RD3L | 1.99 | 10.81 | 1.21 | 1.1 |
| LRRC39 | 1.92 | 10.76 | 1.1 | 1.23 |
| SLC25A4 | 2.33 | 10.73 | 3.77 | 9.45 |
| NEBL | 2.26 | 10.45 | 3.36 | 6.36 |
| FAM155B | 1.6 | 10.34 | 1.02 | 1.11 |
| HJV | 1.58 | 10.24 | NA | NA |
| KLHL41 | 1.15 | 10.21 | 1.67 | 1.27 |
| ASB10 | 1.9 | 9.93 | 1.16 | 1.05 |
| TBX5 | 1.46 | 9.89 | 3.04 | 1.45 |
| METTL7B | 0.96 | 9.85 | 0.15 | 1.02 |
| RBM24 | 1.85 | 9.81 | 2.26 | 1.57 |
| PKP2 | 1.65 | 9.56 | 1.59 | 1.89 |
| KLHL38 | 1.2 | 9.49 | 0.06 | 1 |
| SBK3 | 1.04 | 9.31 | 0.26 | 1.01 |
| CORIN | 1.46 | 9.19 | 1.59 | 1.26 |
| CAVIN4 | 1.48 | 9.16 | NA | NA |
| CAV3 | 2.26 | 8.63 | 0.9 | 1.07 |
| COX7A1 | 2.49 | 8.45 | 7.43 | 50.55 |
| SLC4A3 | 2.3 | 8.27 | 2.78 | 1.82 |
| GATA4 | 1.94 | 7.67 | 0.6 | 1.14 |
| PEBP4 | 1.85 | 7.67 | 0.7 | 1.25 |
| MLIP | 1.43 | 7.14 | 2.02 | 1.65 |
| DOK7 | 1.53 | 7.13 | 0.23 | 1.03 |
| RBM20 | 2.03 | 6.94 | 2 | 1.48 |
| EEF1A2 | 1.87 | 6.78 | 1.28 | 1.35 |
| CHRNE | 0.92 | 6.65 | 0.03 | 0.99 |
| ATP2A2 | 1.73 | 6.64 | 2.08 | 4.52 |
| PLA2G5 | 1.82 | 6.58 | 1.04 | 1.54 |
| PLPP7 | 2.31 | 6.2 | -0.05 | 0.99 |
| FSD2 | 1.74 | 6.11 | 1.56 | 1.23 |
| FLNC | 0.67 | 6.11 | 7.38 | 6.69 |
| CRYAB | 1.3 | 5.93 | 2.47 | 5.66 |
| S100A1 | 2.2 | 5.86 | 0.1 | 0.8 |
| TENM2 | 1.36 | 5.86 | 0.4 | 1.08 |
| TNNI1 | 0.64 | 5.85 | 1.55 | 1.27 |
| PLN | 1.4 | 5.77 | 7.23 | 8.69 |
| CDH2 | 1.68 | 5.73 | 1.74 | 2.01 |
| FHL2 | 1.04 | 5.7 | 0.48 | 1.35 |
| CYP2J2 | 1.6 | 5.67 | 0.52 | 1.17 |
| FGF12 | 1.65 | 5.5 | 1.48 | 1.91 |
| CCDC141 | 1.6 | 5.48 | 1.07 | 1.36 |
| TNNI3K | 1.71 | 5.47 | 0.31 | 1.05 |
| SLC5A1 | 0.89 | 5.47 | 0.88 | 1.24 |
| TNNT1 | 0.74 | 5.31 | 0.63 | 1.4 |
| FHOD3 | 1.52 | 5.29 | 0.23 | 1.05 |
| CLIC5 | 1.62 | 5.1 | 1.58 | 1.74 |
| TPM1 | 1.78 | 5.06 | 3.71 | 17.66 |
| LRRC2 | 1.07 | 5.05 | 1.53 | 1.51 |
| GOT1 | 1.8 | 5.03 | 1.21 | 1.88 |
| DES | 1.2 | 3.88 | 7.99 | 8.88 |
| NEXN | 1.05 | 2.92 | 4.73 | 7.42 |
| MTRNR2L8 | 0.27 | 1.32 | 3.56 | 24.52 |
| MTRNR2L1 | 0.31 | 1.2 | 3.98 | 16.82 |
| MTRNR2L10 | 0.45 | 1.12 | 4.58 | 15.78 |
| MTRNR2L3 | -0.08 | 1 | 6.99 | 7.85 |
| MSRB3 | -0.35 | 0.65 | 4.82 | 8.38 |

### Comparison of 8-mers From Reference Sequences of Cardiac Proteins and SARS-CoV-2 Proteins

The translated proteome from the reference SARS-CoV-2 genome (NC_045512.2) was downloaded from UniProt.^{45,46} A sliding window approach was used to enumerate all 8-mers from the 17 proteins in this viral proteome. Similarly, a sliding window approach was used to generate all 8-mers from the reference amino acid sequences of the previously defined 144 cardiac proteins, including the canonical isoforms and all described isoforms indicated in UniProt. A pairwise comparison of all 8-mers in these two groups was then performed by calculating the Hamming distance using the stringdist function from the stringdist package (version 0.9.8) in R (version 4.0.3). In a control analysis, the same approach was used to calculate the Hamming distance between all SARS-CoV-2 8-mers and the control sets of 369 human proteins enriched in the brain or 198 human proteins enriched in the skin (described above).

### Assessing the Impact of Human and SARS-CoV-2 Variants on Cardiac Peptide Matches

To assess the impact of human genetic variation on potential molecular mimicry, all missense variants were retrieved from the gnomAD database for the previously identified cardiac proteins that had at least one 8-mer similar to peptide in the SARS-CoV-2 reference proteome (Hamming distance = 1).²² The gnomad-api (https://gnomad.broadinstitute.org/api) was used to fetch the variant calls from gnomad_r2_1 version from the Human GRCh37 genome assembly. The variants in this gnomad version (GRCh37/hg19) were derived from 125,748 exome sequences and 15,708 whole-genome sequences from unrelated individuals sequenced as part of various disease-specific and population genetic studies. For any variants that alter the amino acid sequence of a potentially mimicked peptide, whether the mutation resulted in an exact match (Hamming distance = 0) to the corresponding 8-mer from the SARS-CoV-2 reference proteome was determined.

To assess the impact of viral evolution on potential molecular mimicry, the cardiac 8-mers with Hamming distance of 1 (including any alterations of these 8-mers arising from human genetic variation as described above) were queried against all protein variants encoded in 4,854,709 SARS-CoV-2 genomes deposited in the GISAID database (last accessed Nov. 5, 2021).²⁴ Here, it was determined whether any mutations in viral genomes (relative to the reference sequence) resulted in 8-mers which exactly match one or more cardiac peptides.

### Evaluation of Mimicked Peptides for Inclusion in Immune Epitopes

For any 8-mers which showed an exact match between a cardiac peptide (reference or variant sequences) and a SARS-CoV-2 peptide (reference or variant sequences), the 8-mer was queried using the Immune Epitope Database (IEDB; www.iedb.org) and Analysis Resource.²³ Any linear peptide epitope with a Blast similarity of at least 90% from any human host that had positive experimental evidence in any assay (T cell, B cell, or MHC Ligand) was sought out. No MHC class restrictions or disease filters were applied.

### REFERENCES

1. Chung, M. K. et al. COVID-19 and Cardiovascular Disease: From Bench to Bedside. Circ. Res. 128, 1214-1236 (2021).

2. Puntmann, V. O. et al. Outcomes of Cardiovascular Magnetic Resonance Imaging in Patients Recently Recovered From Coronavirus Disease 2019 (COVID-19). JAMA Cardiol 5, 1265-1273 (2020).

3. Barda, N. et al. Safety of the BNT162b2 mRNA Covid-19 Vaccine in a Nationwide Setting. N. Engl. J. Med. 385, 1078-1090 (2021).

4. CDC. Myocarditis and Pericarditis After mRNA COVID-19 Vaccination. https://www.cdc.gov/coronavirus/2019-ncov/vaccines/safety/myocarditis.html (2021).

5. Witberg, G. et al. Myocarditis after Covid-19 Vaccination in a Large Health Care Organization. New England Journal of Medicine (2021) doi:10.1056/nejmoa2110737.

6. Simone, A. et al. Acute Myocarditis Following COVID-19 mRNA Vaccination in Adults Aged 18 Years or Older. JAMA Intern. Med. (2021) doi:10.1001/jamainternmed.2021.5511.

7. Bozkurt, B., Kamat, I. & Hotez, P. J. Myocarditis With COVID-19 mRNA Vaccines. Circulation 144, 471-484 (2021).

8. Proal, A. D. & VanElzakker, M. B. Long COVID or Post-acute Sequelae of COVID-19 (PASC): An Overview of Biological Factors That May Contribute to Persistent Symptoms. Front. Microbiol. 12, 698169 (2021).

9. Galeotti, C. & Bayry, J. Autoimmune and inflammatory diseases following COVID-19. Nature reviews. Rheumatology vol. 16 413-414 (2020).

10. Kanduc, D. From Anti-SARS-CoV-2 Immune Responses to COVID-19 via Molecular Mimicry. Antibodies (Basel) 9, (2020).

11. Gowthaman, U. & Eswarakumar, V. P. Molecular mimicry: good artists copy, great artists steal. Virulence 4, 433-434 (2013).

12. Cusick, M. F., Libbey, J. E. & Fujinami, R. S. Molecular mimicry as a mechanism of autoimmune disease. Clin. Rev. Allergy Immunol. 42, 102-111 (2012).

13. Oldstone, M. B. A. Molecular Mimicry, Microbial Infection, and Autoimmune Disease: Evolution of the Concept. in Molecular Mimicry: Infection-Inducing Autoimmune Disease (ed. Oldstone, M. B. A.) 1-17 (Springer Berlin Heidelberg, 2005).

14. Adderson, E. E., Shikhman, A. R., Ward, K. E. & Cunningham, M. W. Molecular analysis of polyreactive monoclonal antibodies from rheumatic carditis: human anti-N-acetylglucosamine/anti-myosin antibody V region genes. J. Immunol. 161, 2020-2031 (1998).

15. Cunningham, M. W., Antone, S. M., Smart, M., Liu, R. & Kosanke, S. Molecular analysis of human cardiac myosin-cross-reactive B- and T-cell epitopes of the group A streptococcal M5 protein. Infect. Immun. 65, 3913-3923 (1997).

16. Cunningham, M. W. Autoimmunity and molecular mimicry in the pathogenesis of post-streptococcal heart disease. Front. Biosci. 8, s533-43 (2003).

17. GTEx Consortium. The Genotype-Tissue Expression (GTEx) project. Nat. Genet. 45, 580-585 (2013).

18. Venkatakrishnan, A. J. et al. Knowledge synthesis of 100 million biomedical documents augments the deep expression profiling of coronavirus receptors. Elife 9, (2020).

19. Rozenblatt-Rosen, O., Stubbington, M. J. T., Regev, A. & Teichmann, S. A. The Human Cell Atlas: from vision to reality. Nature 550, 451-453 (2017).

20. Han, X. et al. Construction of a human cell landscape at single-cell level. Nature 581, 303-309 (2020).

21. UniProt Consortium. UniProt: the universal protein knowledgebase in 2021. Nucleic Acids Res. 49, D480-D489 (2021).

22. Karczewski, K. J. et al. The mutational constraint spectrum quantified from variation in 141,456 humans. Nature 581, 434-443 (2020).

23. Vita, R. et al. The Immune Epitope Database (IEDB): 2018 update. Nucleic Acids Res. 47, D339-D343 (2019).

24. Shu, Y. & McCauley, J. GISAID: Global initiative on sharing all influenza data - from vision to reality. Euro Surveill. 22, (2017).

25. IEDB.org: Free epitope database and prediction resource. https://www.iedb.org/home_v3.php.

26. Thorsen, K. et al. Loss-of-activity-mutation in the cardiac chloride-bicarbonate exchanger AE3 causes short QT syndrome. Nat. Commun. 8, 1696 (2017).

27. Anion exchange protein 3. https://www.uniprot.org/uniprot/P48751.

28. Altschul, S. F., Gish, W., Miller, W., Myers, E. W. & Lipman, D. J. Basic local alignment search tool. Journal of Molecular Biology vol. 215 403-410 (1990).

29. Ehrenfeld, M. et al. Covid-19 and autoimmunity. Autoimmunity Reviews vol. 19 102597 (2020).

30. Anand, P., Puranik, A., Aravamudan, M., Venkatakrishnan, A. J. & Soundararajan, V. SARS-CoV-2 strategically mimics proteolytic activation of human ENaC. Elife 9, (2020).

31. Venkatakrishnan, A. J. et al. Benchmarking evolutionary tinkering underlying human-viral molecular mimicry shows multiple host pulmonary-arterial peptides mimicked by SARS-CoV-2. Cell Death Discov 6, 96 (2020).

32. Corbett, K. S. et al. SARS-CoV-2 mRNA vaccine design enabled by prototype pathogen preparedness. Nature 586, 567-571 (2020).

33. Walsh, E. E. et al. Safety and Immunogenicity of Two RNA-Based Covid-19 Vaccine Candidates. N. Engl. J. Med. 383, 2439-2450 (2020).

34. Smatti, M. K. et al. Viruses and Autoimmunity: A Review on the Potential Interaction and Molecular Mechanisms. Viruses 11, (2019).

35. Fujinami, R. S., von Herrath, M. G., Christen, U. & Whitton, J. L. Molecular mimicry, bystander activation, or viral persistence: infections and autoimmune disease. Clin. Microbiol. Rev. 19, 80-94 (2006).

36. Sewell, A. K. Why must T cells be cross-reactive? Nat. Rev. Immunol. 12, 669-677 (2012).

37. Borbulevych, O. Y., Santhanagopolan, S. M., Hossain, M. & Baker, B. M. TCRs used in cancer gene therapy cross-react with MART-⅟Melan-A tumor antigens via distinct mechanisms. J. Immunol. 187, 2453-2463 (2011).

38. Scott, D. R., Borbulevych, O. Y., Piepenbrink, K. H., Corcelli, S. A. & Baker, B. M. Disparate degrees of hypervariable loop flexibility control T-cell receptor cross-reactivity, specificity, and binding mechanism. J. Mol. Biol. 414, 385-400 (2011).

39. Garcia, K. C. et al. Structural basis of plasticity in T cell receptor recognition of a self peptide-MHC antigen. Science 279, 1166-1172 (1998).

40. Wooldridge, L. et al. A single autoimmune T cell receptor recognizes more than a million different peptides. J. Biol. Chem. 287, 1168-1177 (2012).

41. Wang, L. et al. Single-cell reconstruction of the adult human heart during heart failure and recovery reveals the cellular landscape underlying cardiac function. Nat. Cell Biol. 22, 108-119 (2020).

42. Doddahonnaiah, D. et al. A Literature-Derived Knowledge Graph Augments the Interpretation of Single Cell RNA-seq Datasets. Genes 12, (2021).

43. Stuart, T. et al. Comprehensive Integration of Single-Cell Data. Cell 177, 1888-1902.e21 (2019).

44. Wolock, S. L., Lopez, R. & Klein, A. M. Scrublet: Computational identification of cell Doublets in Single-cell transcriptomic data. Cell Syst. 8, 281-291.e9 (2019).

45. UniProt.https://covid-19.uniprot.org/uniprotkb?query=*&facets=other_organism:Severe%20acute%20respiratory %20syndrome%20coronavirus%202.

46. Severe acute respiratory syndrome coronavirus 2 isolate Wuhan-Hu-1, co - Nucleotide -NCBI. https://www.ncbi.nlm.nih.gov/nuccore/NC_045512.2.

What is claimed is:

1. A method of treating a coronavirus infection in a subject in need thereof, comprising
a) determining whether the subject is susceptible to an autoimmune inflammatory reaction by
i) identifying candidate genes that are overexpressed in a target tissue or organ in the subject;
ii) preparing a plurality of 8-mer peptide sequences encoded by the overexpressed candidate genes;
iii) identifying at least one 8-mer peptide of the overexpressed candidate genes that is an identical match to a corresponding 8-mer viral peptide; and
b) administering to said subject an autoimmune therapeutic.

2. A method of treating or preventing cardiac injury in a subject infected with coronavirus, the method comprising identifying the presence of at least one mimicked peptide set forth in Table 2, mutated cardiac peptide set forth in Table 3, or any combination thereof in a sample from the subject; wherein the presence of any one or more of the peptides set forth in Table 2 or mutated cardiac peptides set forth in Table 3 indicates whether the subject is susceptible to cardiac injury, and treating said patient for cardiac injury.

3. A method of treating a coronavirus infection in a subject in need thereof, comprising
a) identifying the presence of at least one mimicked peptide set forth in Table 2, mutated cardiac peptide set forth in Table 3, or any combination thereof in a sample from the subject; and
b) administering to said subject a therapeutic to the subject.

4. The method of claim 3, wherein the therapeutic is selected from remdesivir, antibody therapy, dexamethasone, convalescent plasma, ritonavir, and molnupiravir.

5. The method of claim 4, wherein the antibody therapy comprises one or more of sotrovimab, bamlanivimab, etesevimab, casirivimab, and imdevimab.
